(19) United States
(12) Patent Application Publication
(10) Pub. No.: US 2015/0258310 A1
(43) Pub. Date: Sep. 17, 2015

(54) CABLE CONTROL SYSTEM

(71) Applicant: Michel Chaim Cohn, Weston, FL (US)

(72) Inventor: Michel Chaim Cohn, Weston, FL (US)

(21) Appl. No.: 14/211,735

(22) Filed: Mar. 14, 2014

(51) Int. Cl. A61M 25/09

(52) U.S. Cl. CPC A61M 25/09041; A61M 2025/09116; A61M 2025/09133

(57) ABSTRACT

A cable control system enhances controllability of a cable through the use of torque, friction force, and adherence forces. The system includes a sheath portion configured to grip the cable in a manner that provides enhanced controllability and manipulation of the cable. The sheath portion can include a glove or a finger sheath. An actuating member inserts into the sheath portion and applies a normal force to a pivot point along the sheath portion. The normal force includes a twist, rotation, push, and pull. This application of the normal force provides torque to the cable, which enables enhanced control over the cable, especially in tortuous cavities. An outer surface utilizes a surface texture having a generally nonplanar surface, and configured to resist lateral movement between the outer surface and the cable. The outer surface further comprises a hydrophobic surface for increasing adherence to a cable coated with polar solvent.

### FIELD OF THE INVENTION

The present invention relates generally to a cable control system. More so, a cable control system enhances control of a cable through torque, friction force, and at least partial adherence to the cable.

### BACKGROUND OF THE INVENTION

The following background information may present examples of specific aspects of the prior art (e.g., without limitation, approaches, facts, or common wisdom) that, while expected to be helpful to further educate the reader as to additional aspects of the prior art, is not to be construed as limiting the present invention, or any embodiments thereof, to anything stated or implied therein or inferred thereupon.

Typically, a cable is a thin, usually flexible wire. A guide wire is a type of cable that can be inserted into a confined or tortuous cavity to act as a guide for subsequent insertion of a stiffer or bulkier instrument. A medical cable is commonly used for a variety of medical procedures. Such procedures include angioplasty, stenting, pacemaker insertion, electrophysiology studies, atherectomy, and thrombolysis. These procedures require intrusive operations in an often slippery, tortuous, and narrow body cavity.

Often, to maneuver a cable at a desired location within a patient, a medical professional navigates the cable through the patient's cavities and spaces by manipulating the cable. Such manipulation includes advancing of the cable into a patient's vasculature or other portion of the patient's body while applying torque to the cable.

To manipulate the cable, medical professionals have traditionally used devices which require two-handed operability. As the cable is advanced into the cavity the distance between the patient's body and the torque device decreases. When the proximity between the patient's body and the torque device decreases, the medical professional may loosen the torque device, reposition the torque device proximally along the cable to provide an additional length of cable between the patient's body and the torque device, and then tighten the torque device to secure its position along the length of the cable. The process of loosening and repositioning the torque device may be repeated several times during the placement of the cable.

Due to the complexities of some cable placement procedures, it can be inconvenient or impractical for a practitioner to utilize both hands to thread the cable through the catheter or reposition the torque device along the length of the cable. Also, the slippery nature of the body cavities reduces torque on the cable. Additionally, some devices do not provide adequate gripping of the cable as may be required to push the cable through a vascular lesion or other guidewire path occlusion. Where an occlusion is encountered, the practitioner may over tighten the device in a manner that causes damage to the cable.

In view of the foregoing, it is clear that these traditional techniques are not perfect and leave room for more optimal approaches.

### SUMMARY OF THE INVENTION

This invention is directed to a cable control system that is configured to enhance controllability of a cable through the use of forces, such as torque, friction force, and adherence forces. The system is configured to enable one-handed gripping of the cable, and provide torque and adhesive friction to the cable. In one embodiment, the cable may include a thin, usually flexible wire. Due to the flexible nature of the cable, torque and frictional forces can enhance the grip and control on the cable. In some embodiments, the system may be disposed to controllably maneuver the cable from inside or outside a cavity. In either case, the system is configured such that torque, friction force, and adherence forces are applied to the cable, which works to enhance controllability and manipulation of the cable.

In some embodiments, the system provides torque to the cable such that control and grip are enhanced. For example, applying torque to the cable may enable the cable to change the spatial orientation of a cable terminal point when negotiating a tortuous or slippery cavity. The torque is produced when a normal force is applied to a pivot point on the system. The normal force is rotatably applied about the pivot point to create torque on the cable. The system may also impart friction forces on the cable through an outer surface having a surface texture. The surface texture engages and moves relative to the cable to generate the friction force. The system may also increase adherence to the cable through a hydrophobic surface that counters a cable that is coated with a polar solvent.

In some embodiments, the system may include a sheath portion configured to control the cable. The sheath portion includes an inner surface for receiving, and forming a unitary body with an actuating member. The actuating member may include a finger or hand that, while integrated with the sheath portion, applies a normal force to a pivot point along the sheath portion. The normal force may be rotationally applied to the pivot point to provide torque to the cable. In this manner, the cable may be maneuvered and controlled more effectively.

In some embodiments, sheath portion comprises an outer surface configured to engage the cable. The outer surface includes a surface texture having friction enhancing characteristics, such as roughness, irregularities, and nonplanar topographies. The surface texture resists lateral movement between the outer surface and the cable. The resistance of lateral movement generates traction and enhances control of the cable. The outer surface may further include a hydrophobic surface that increase adherence to the cable, especially a slippery cable operating in a wet cavity. In this manner, a torque, a friction force, and a hydrophobic surface work together to enhance control of the cable.

A first aspect of the present invention provides a cable control system that enhances control of a cable, the cable control system comprising:

a sheath portion configured to control a cable,

the sheath portion comprising an inner surface configured to receive an actuating member, the actuating member configured to apply a normal force on the sheath portion,

the sheath portion further comprising a pivot point,

wherein the normal force engages the pivot point to provide torque to the cable,

the sheath portion further comprising an outer surface configured to engage the cable,

the outer surface comprising a surface texture, the surface texture configured to generate a friction force by moving relative to the cable,

the outer surface further comprising a substantially hydrophobic surface, the substantially hydrophobic surface configured to adhere to the cable,

wherein the torque, the friction force, and the adherence to the cable enable enhanced controllability of the cable.

In a second aspect, the system provides sufficient torque and adhesive forces to facilitate advancement of the cable through a narrow and tortuous cavity.

In another aspect, the system is disposed to position outside or inside the cavity while maneuvering the cable.

In another aspect, the cable comprises a medical guide wire.

In another aspect, the actuating member comprises a finger or a hand.

In another aspect, the sheath portion comprises a stretchable material configured to conform to the size and dimension of the actuating member.

In another aspect, the actuating member dons the sheath portion for engaging the cable.

In another aspect, the sheath portion comprises a glove or at least one finger sheath.

In another aspect, the actuating member comprises a finger or a hand.

In another aspect, the pivot point comprises a terminal point of the actuating member.

In another aspect, the terminal point comprises a finger tip.

In another aspect, the surface texture comprises a gauge fiber texture, and/or a protrusion texture, and/or a ridge texture, and/or a rough texture.

In another aspect, the hydrophobic surface comprises a non-polar molecular structure that resists polar solvents on the cable.

These and other advantages of the invention will be further understood and appreciated by those skilled in the art by reference to the following written specification, claims and appended drawings.

### BRIEF DESCRIPTION OF THE DRAWINGS

The present invention is illustrated by way of example, and not by way of limitation, in the figures of the accompanying drawings and in which like reference numerals refer to similar elements and in which:

FIG. 1 illustrates a detailed perspective view of an exemplary cable control system, in accordance with an embodiment of the present invention;

FIG. 2 illustrates a detailed perspective view of an exemplary actuating member forming a unitary body with an exemplary cable control system to control an exemplary cable, in accordance with an embodiment of the present invention;

FIGS. 3A, 3B, 3C, and 3D illustrate exemplary surface textures on an outer surface, where FIG. 3A illustrates a gauze fiber pattern, FIG. 3B illustrates a ridged pattern, FIG. 3C illustrates a protrusion pattern, and FIG. 3D illustrates a rough pattern, in accordance with an embodiment of the present invention; and

FIG. 4 illustrates flowchart diagram of an exemplary method for controlling a cable, in accordance with an embodiment of the present invention.

Like reference numerals refer to like parts throughout the various views of the drawings.

### DETAILED DESCRIPTION OF THE INVENTION

The present invention is best understood by reference to the detailed figures and description set forth herein.

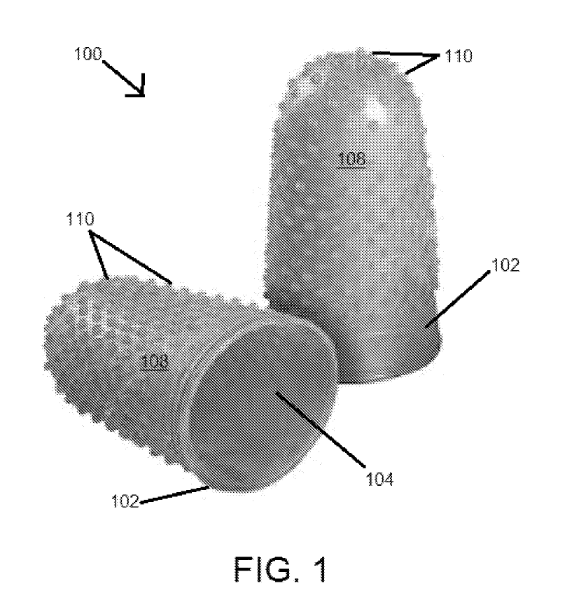

The following detailed description is merely exemplary in nature and is not intended to limit the described embodiments or the application and uses of the described embodiments. As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure, which is defined by the claims. For purposes of description herein, the terms “upper,” “lower,” “left,” “rear,” “right,” “front,” “vertical,” “horizontal,” and derivatives thereof shall relate to the invention as oriented in FIG. 1. Furthermore, there is no intention to be bound by any expressed or implied theory presented in the preceding technical field, background, brief summary or the following detailed description. It is also to be understood that the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise.

It is to be further understood that the present invention is not limited to the particular methodology, compounds, materials, manufacturing techniques, uses, and applications, described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to “an element” is a reference to one or more elements and includes equivalents thereof known to those skilled in the art. Similarly, for another example, a reference to “a step” or “a means” is a reference to one or more steps or means and may include sub-steps and subservient means. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that may be construed to express approximation should be so understood unless the context clearly dictates otherwise.

Unless defined otherwise, all technical and scientific terms used herein have the same meanings as commonly understood by one of ordinary skill in the art to which this invention belongs. Preferred methods, techniques, devices, and materials are described, although any methods, techniques, devices, or materials similar or equivalent to those described herein may be used in the practice or testing of the present invention. Structures described herein are to be understood also to refer to functional equivalents of such structures. The present invention will now be described in detail with reference to embodiments thereof as illustrated in the accompanying drawings.

FIG. 1 illustrates a cable control system 100 that is configured to enhance controllability of a cable 112 through the use of forces, such as torque, friction force, and adherence forces. The system 100 includes a sheath portion 102 configured to grip the cable 112 in a manner that provides enhanced controllability and manipulation of the cable 112. The sheath portion 102 may include, without limitation, a glove or at least one finger sheath. Suitable materials for the sheath portion 102 may include, without limitation, latex, rubber, polymers, animal skin, synthetic fabrics, and fiber gauze.

In some embodiments, the sheath portion 102 may comprise an inner surface 104 configured to receive an actuating member 114. The actuating member 114 may include, without limitation, a finger, a hand, a robotic limb, and a rod. The actuating member 114 may be configured to apply a normal force on the sheath portion 102. The normal force may include the actuating member 114 forming a grip around the cable 112 while twisting, rotating, pushing, and pulling. The sheath portion 102 further includes a pivot point 106 that positions at the terminal ends of the actuating member 114. The normal force engages the pivot point 106 to provide torque to the cable 112. The torque enables the cable 112 to maneuver in a more controllable manner, especially in a slippery or tortuous cavity.

In some embodiments, the sheath portion 102 may further comprise an outer surface 108 configured to engage the cable 112. The outer surface 108 comprises a surface texture 110 configured to generate a friction force by moving relative to the cable 112. The surface texture 110 is configured to resist lateral movement between the outer surface 108 and the cable 112. The resistance of lateral movement generates traction and enhances control of the cable 112. The surface texture 110 comprises a shape and texture efficacious for generating friction. The surface texture 110 may include, without limitation, a gauze fiber texture 116 having gauze fiber content; a ridge texture 118 having parallel ridges; a protrusion texture 120 having a plurality of protrusions; and a rough texture 122 having a generally rough, nonplanar surface. However in other embodiments, any texture may be utilized that creates adhesive forces between the outer surface 108 and the cable 112.

The outer surface 108 may further include a hydrophobic surface (not shown) configured, such that frictional adhesiveness is created between the outer surface 108 and the cable 112. The hydrophobic surface comprises a non-polar molecular structure that resists polar solvents on the cable 112. The hydrophobic surface helps increase adherence to the cable 112, especially a slippery cable 112 operating in a wet cavity. In this manner, the torque, the friction force, and the adherence to the cable 112 allow for enhanced controllability of the cable 112.

Those skilled in the art will recognize that additional factors that may be determinative of the amount of friction generated between the outer surface 108 and the cable 112 may include, without limitation, magnitude of the normal force, velocity of movement between the outer surface 108 and the cable 112, lubrication, materials of the sheath portion 102, friction coefficient, and temperature.

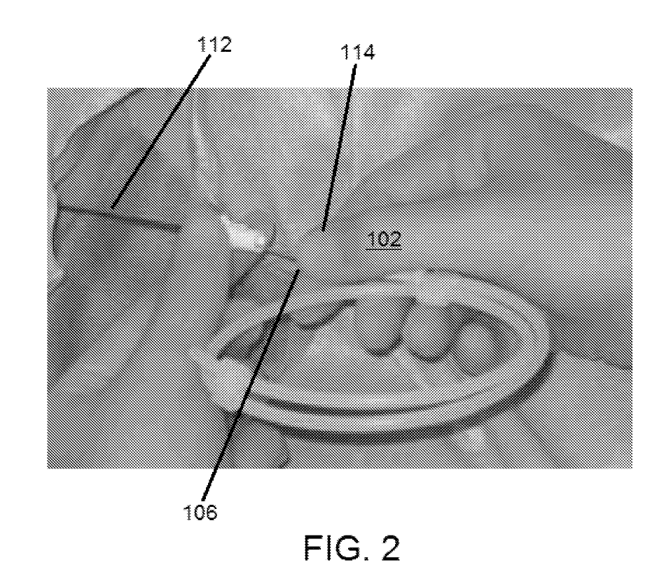

Turning now to FIG. 2, the system 100 is configured to enable one-handed gripping of the cable 112, and provide torque and adhesive friction to the cable 112. In one embodiment, the cable 112 may include a thin, usually flexible wire. Due to the flexible nature of the cable 112, torque and frictional forces can enhance the grip and control on the cable 112. In some embodiments, the system 100 may be disposed to controllably maneuver the cable 112 from inside or outside a cavity. In either case, the system 100 is configured such that torque, friction force, and adherence forces are applied to the cable 112, which works to enhance controllability and manipulation of the cable 112.

Those skilled in the art will recognize that a body cavity may include a smooth, slippery surface that does not provide adhesive characteristics. Additionally, the body cavity may be tortuous and have barriers, such as vascular lesions and other cable path occlusions. A hydrophilic object passing through such a cavity may not have sufficient adhesive traction to generate torque for maneuvering through the body cavity. Additionally, the system 100 must also provide a sufficient grip on the cable 112, and in some cases when the cable 112 is manipulated by a doctor, enable the doctor to maintain a free hand during an operation.

In some embodiments, the system 100 provides torque to the cable 112 such that control and grip are enhanced. For example, applying torque to the cable 112 may enable the cable 112 to change the spatial orientation of a cable terminal point when negotiating a tortuous or slippery cavity. The torque is produced when a normal force is applied to a pivot point 106 on the system 100. The normal force is rotatably applied about the pivot point 106 to create torque on the cable 112. The system 100 may also impart friction forces on the cable 112 through an outer surface 108 that utilizes a surface texture 110. The surface texture 110 engages and moves relative to the cable 112 to generate the friction force. The system 100 may also increase adherence to the cable 112 through the hydrophobic surface.

In some embodiments, the system 100 may include a sheath portion 102 configured to grip and control the cable 112. The sheath portion 102 may include an inner surface 104 for receiving, and forming a unitary body with an actuating member 114. The actuating member 114 may include a finger or hand which, while integrated with the sheath portion 102, applies a normal force to a pivot point 106 along the sheath portion 102. The normal force may be rotationally applied to the pivot point 106 to provide torque to the cable 112. In this manner, the cable 112 may be maneuvered and controlled more effectively.

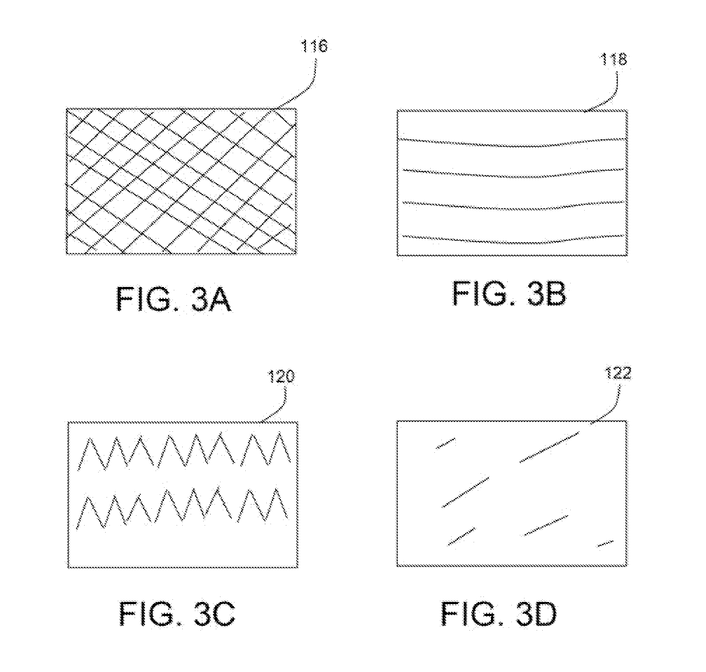

Turning now to FIGS. 3A-3D, the sheath portion 102 comprises an outer surface 108 configured to engage the cable 112. The outer surface 108 includes a surface texture 110 having friction enhancing characteristics, such as roughness, irregularities, and nonplanar topographies. The surface texture 110 resists lateral movement between the outer surface 108 and the cable 112. The resistance of lateral movement generates traction and enhances control of the cable 112. The surface texture 110 may include, without limitation, a gauze fiber texture 116 having gauze fiber content; a ridge texture 118 having parallel ridges; a protrusion texture 120 having a plurality of protrusions; and a rough texture 122 having a generally rough, nonplanar surface. However in other embodiments, any texture may be utilized that creates adhesive forces between the outer surface 108 and the cable 112.

The outer surface 108 may further include a hydrophobic surface that increase adherence to the cable 112, especially a slippery cable 112 operating in a wet or slippery cavity. In this manner, a torque, a friction force, and a hydrophobic surface work together to enhance control of the cable 112.

In one alternative embodiment, the sheath portion 102 may include an image capturing device (not shown), such as a camera. The image capturing device may help guide an operator of the system 100, whereby the sheath portion may be maneuvered to create torque, change of direction, and linear movements based on the image. The image capturing device may include an illumination portion (not shown) for enhanced visibility.

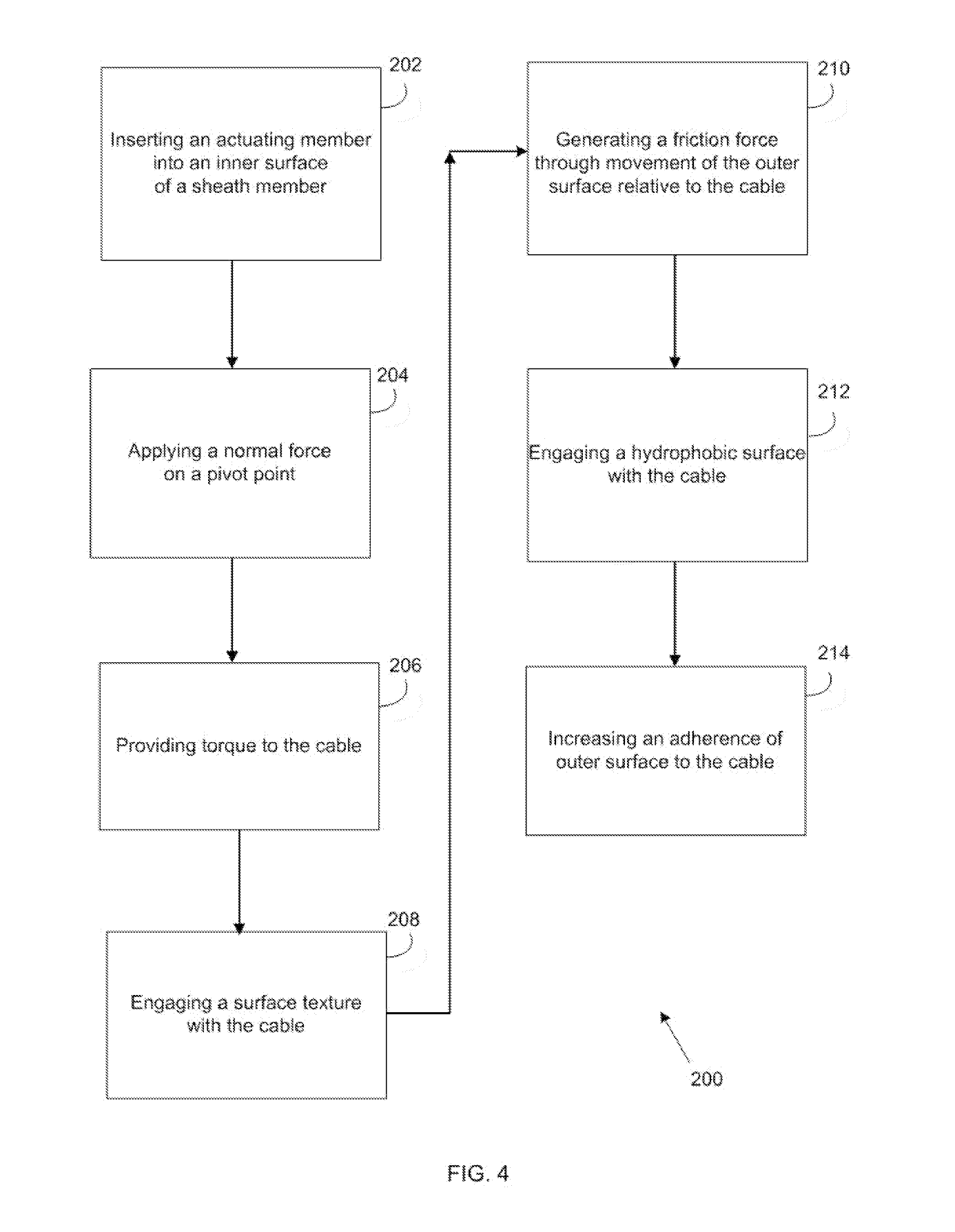

FIG. 4 illustrates a flowchart diagram of an exemplary method 200 for controlling a cable 112. The method utilizes a cable control system 100 to enhance controllability of a cable 112 through the use of various forces, including, without limitation, torque, friction force, and increased adherence to the cable 112. The system 100 includes a sheath portion 102 configured to grip the cable 112 in a manner that provides enhanced controllability and manipulation of the cable 112.

The method may include an initial Step 202 of inserting an actuating member 114 into an inner surface 104 of a sheath portion 102. The actuating member 114 may pass through an aperture in the sheath portion 102, forming a unitary body thereto. The actuating member 114 may include a finger or hand that, while integrated with the sheath portion 102, applies a normal force to a pivot point 106 along the sheath portion 102. The method 200 may further comprise a Step 204 of applying a normal force on a pivot point 106. The pivot point 106 may be located at a terminal point of the actuating member 114, such as a finger tip. The torque is produced when a normal force is applied to the pivot point 106. A Step 206 includes providing torque to the cable 112. The normal force is rotatably applied about the pivot point 106 to create torque on the cable 112, such that control and grip on the cable 112 are enhanced. For example, applying torque to the cable 112 may enable the cable 112 to change the spatial orientation of a cable terminal point when negotiating a tortuous or slippery cavity.

In some embodiments, a Step 208 comprises engaging a surface texture 110 with the cable 112. The surface texture 110 comprises a shape and texture efficacious for generating friction. The surface texture 110 may comprise friction enhancing characteristics, such as roughness, irregularities, and nonplanar topographies. A Step 210 includes generating a friction force through movement of an outer surface 108 relative to the cable 112. The outer surface 108 configured to engage the cable 112, and may include a surface texture 110 configured to generate a friction force by moving relative to the cable 112.

In some embodiments, a Step 212 may include engaging a hydrophobic surface with the cable 112. The outer surface 108 may further include a hydrophobic surface configured, such that frictional adhesiveness is created between the outer surface 108 and the cable 112. A final Step 214 includes increasing an adherence of the outer surface 108 to the cable 112. The hydrophobic surface comprises a non-polar molecular structure that resists polar solvents on the cable 112. The hydrophobic surface helps increase adherence to the cable 112, especially a slippery cable 112 operating in a wet cavity. In this manner, the torque, the friction force, and the adherence to the cable 112 provide enhanced controllability of the cable 112.

Since many modifications, variations, and changes in detail can be made to the described preferred embodiments of the invention, it is intended that all matters in the foregoing description and shown in the accompanying drawings be interpreted as illustrative and not in a limiting sense. Thus, the scope of the invention should be determined by the appended claims and their legal equivalence.

Claim elements and steps herein may have been numbered and/or lettered solely as an aid in readability and understanding. Any such numbering and lettering in itself is not intended to and should not be taken to indicate the ordering of elements and/or steps in the claims.

The corresponding structures, materials, acts, and equivalents of all means or step plus function elements in the claims below are intended to include any structure, material, or act for performing the function in combination with other claimed elements as specifically claimed.

The Abstract is provided to comply with 37 C.F.R. Section 1.72(b) requiring an abstract that will allow the reader to ascertain the nature and gist of the technical disclosure. It is submitted with the understanding that it will not be used to limit or interpret the scope or meaning of the claims. The following claims are hereby incorporated into the detailed description, with each claim standing on its own as a separate embodiment.

What I claim is:

1. A cable control system for enhancing control of a cable, the cable control system comprising:
a sheath portion configured to control a cable,
the sheath portion comprising an inner surface configured to receive an actuating member, the actuating member configured to apply a normal force on the sheath portion,
the sheath portion further comprising a pivot point,
wherein the normal force engages the pivot point to provide torque to the cable,
the sheath portion further comprising an outer surface configured to engage the cable,
the outer surface comprising a surface texture, the surface texture configured to generate a friction force by moving relative to the cable,
the outer surface further comprising a substantially hydrophobic surface, the substantially hydrophobic surface configured to adhere to the cable,
wherein the torque, the friction force, and the adherence to the cable enable enhanced controllability of the cable.

2. The system of claim 1, in which the system provides sufficient torque and adhesive forces to facilitate advancement of the cable through a narrow and tortuous cavity.

3. The system of claim 2, in which the system is disposed to position outside or inside the cavity while maneuvering the cable.

4. The system of claim 3, in which the cable comprises a medical guide wire.

5. The system of claim 4, in which the actuating member comprises a finger or a hand.

6. The system of claim 5, in which the sheath portion comprises a stretchable material configured to conform to the size and dimension of the actuating member.

7. The system of claim 6, in which the actuating member dons the sheath portion for engaging the cable.

8. The system of claim 7, in which the sheath portion comprises a glove or at least one finger sheath.

9. The system of claim 8, in which the actuating member comprises a finger.

10. The system of claim 9, in which the pivot point comprises a terminal point of the actuating member.

11. The system of claim 10, in which the terminal point comprises a finger tip.

12. The system of claim 11, in which the surface texture comprises a gauge fiber texture, and/or a protrusion texture, and/or a ridge texture, and/or a rough texture.

13. The system of claim 12, in which the hydrophobic surface comprises a non-polar molecular structure that resists polar solvents on the cable.

14. A cable control method for enhancing control of a cable, the method comprising:
inserting an actuating member into an inner surface of a sheath portion;
applying a normal force on a pivot point;
providing torque to a cable;
engaging a surface texture with the cable;
generating a friction force through movement of an outer surface relative to the cable;
engaging a hydrophobic surface with the cable; and
increasing an adherence of the outer surface to the cable.

15. A cable control system for enhancing control of a cable, the cable control system comprising:
a sheath portion configured to control a cable, the cable comprising a guide wire, the sheath portion comprising a glove,
the sheath portion comprising an inner surface configured to receive an actuating member, the actuating member configured to apply a normal force on the sheath portion, the actuating member comprising a hand,
the sheath portion further comprising a pivot point,
wherein the normal force engages the pivot point to provide torque to the cable,
the sheath portion further comprising an outer surface configured to engage the cable,
the outer surface comprising a surface texture, the surface texture configured to generate a friction force by moving relative to the cable, the surface texture comprising a gauge fiber texture, and/or a ridged texture, and/or a protrusion texture, and/or a rough texture,
the outer surface further comprising a substantially hydrophobic surface, the substantially hydrophobic surface configured to adhere to the cable,
wherein the torque, the friction force, and the adherence to the cable enable enhanced controllability of the cable.

16. The system of claim 15, in which the system provides sufficient torque and adhesive forces to facilitate advancement of the cable through a narrow and tortuous cavity.

17. The system of claim 16, in which the sheath portion comprises a stretchable material configured to conform to the size and dimension of the actuating member.

18. The system of claim 17, in which the pivot point comprises a terminal point of the actuating member.

19. The system of claim 18, in which the terminal point comprises a finger tip.

20. The system of claim 19, in which the hydrophobic surface comprises a non-polar molecular structure that resists polar solvents on the cable.
